(19) United States
(12) Patent Application Publication
(10) Pub. No.: US 2023/0248552 A1
(43) Pub. Date: Aug. 10, 2023

(54) STENT INCLUDING ANTI-MIGRATION MEMBERS

(71) Applicant: BOSTON SCIENTIFIC SCIMED, INC., Maple Grove, MN (US)

(72) Inventors: MICHAEL WALSH, Galway (IE); GARRETT CASSERLY, Galway (IE); KEVIN WINDHEUSER, Hopkinton, MA (US); MOLLY SOLOMON, Groton, MA (US); JASMINE CLEVENGER, Sherborn, MA (US); JONATHAN ROOT, Townsend, MA (US)

(73) Assignee: BOSTON SCIENTIFIC SCIMED, INC., Maple Grove, MN (US)

(21) Appl. No.: 18/164,530

(22) Filed: Feb. 3, 2023

Related U.S. Application Data
(60) Provisional application No. 63/306,733, filed on Feb. 4, 2022.

(51) Int. Cl. A61F 2/848

(52) U.S. Cl. CPC A61F 2/848

(57) ABSTRACT

Example expandable stent designs are disclosed. An example expandable stent includes a tubular scaffold including an inner surface, an outer surface and a lumen extending therein. The expandable stent also includes an outer layer disposed along the outer surface of the tubular scaffold and a first preformed flap attached to the outer layer. Additionally, the preformed flap is configured to releasably engage a vessel wall when the stent is implanted therein.

### CROSS-REFERENCE TO RELATED APPLICATIONS

This application claims the benefit of U.S. Provisional Patent Application Ser. No. 63/306,733, filed Feb. 4, 2022, which is incorporated herein by reference.

### TECHNICAL FIELD

The present disclosure pertains to medical devices, methods for manufacturing medical devices, and the use thereof. More particularly, the present disclosure pertains to stents including anti-migration members, and methods for manufacturing and using such stents.

### BACKGROUND

Implantable medical devices (e.g., expandable stents) may be designed to provide a pathway for digested material, blood, or other fluid to flow therethrough following a medical procedure. Further, some implantable medical devices may incorporate features that aid in fistula treatment, bypass procedures and/or anastomosis treatment. These medical devices may include radially or self-expanding stents which may be implanted transluminally via an endoscope. Additionally, some stents may be implanted in a variety of body lumens such as the esophageal tract, the gastrointestinal tract (including the intestine, stomach and the colon), tracheobronchial tract, urinary tract, biliary tract, vascular system, etc.

In some instances, it may be desirable to design a stent which includes sufficient radial strength to maintain its position within a body lumen while also having the ability to function as a passageway for food or other digested material to flow therethrough. However, in some stents, the compressible and flexible properties that assist in stent positioning may also result in a stent that may migrate from its originally deployed position. For example, stents that are designed to be positioned in the esophageal or gastrointestinal tract may migrate due to peristalsis (i.e., the involuntary constriction and relaxation of the muscles of the esophagus, intestine, and colon which push the contents of the canal therethrough). Additionally, the moist and inherently lubricious environment of the esophagus, intestine, colon, etc. further contributes to a stent's tendency to migrate when deployed therein. However, while it is important to design stents that reduce the degree to which a stent migrates within a body lumen, it also important to design stents that may be easily removed and/or re-positioned from the body lumen post-deployment.

Accordingly, it may be desirable to design a stent which includes an anti-migration structure to mitigate the ability of the stent to migrate in the anatomy. Examples of medical devices including flexible anchoring members are disclosed herein.

### BRIEF SUMMARY

This disclosure provides design, material, manufacturing method, and use alternatives for medical devices. An example expandable stent includes a tubular scaffold including an inner surface, an outer surface and a lumen extending therein. The expandable stent also includes an outer layer disposed along the outer surface of the tubular scaffold and a first preformed flap attached to the outer layer. Additionally, the preformed flap is configured to releasably engage a vessel wall when the stent is implanted therein.

Alternatively or additionally to any of the embodiments above, wherein the first preformed flap is configured to pivot between a radially extended position and a collapsed position.

Alternatively or additionally to any of the embodiments above, wherein the first preformed flap is configured to deflect radially inward from a radially extended position.

Alternatively or additionally to any of the embodiments above, wherein the first preformed flap includes a first attachment region positioned along a first edge of the flap, and wherein the first preformed flap is attached to the outer layer along the first attachment region.

Alternatively or additionally to any of the embodiments above, wherein the first preformed flap is bonded to the outer layer using a bonding material.

Alternatively or additionally to any of the embodiments above, wherein the outer layer, the first preformed flap and the bonding material are formed from the same material.

Alternatively or additionally to any of the embodiments above, further comprising a second preformed flap, and wherein the first preformed flap and the second preformed flap are uniformly spaced around the tubular scaffold.

Alternatively or additionally to any of the embodiments above, wherein the first preformed flap and the second preformed flap are longitudinally aligned with each other along the outer layer of the stent.

Alternatively or additionally to any of the embodiments above, wherein the first preformed flap and the second preformed flap are longitudinally offset from each other along the outer layer of the stent.

Alternatively or additionally to any of the embodiments above, wherein the first preformed flap is positioned along a proximal end region of the tubular scaffold, and wherein the second preformed flap is positioned along a distal end region of the tubular scaffold.

Alternatively or additionally to any of the embodiments above, wherein the first preformed flap extends in a distal direction toward a distal end of the tubular scaffold, and wherein the second preformed flap extends in a proximal direction toward a proximal end of the tubular scaffold.

Alternatively or additionally to any of the embodiments above, further comprising a second preformed flap attached to the outer layer of the stent, a third preformed flap attached to the outer layer of the stent and a fourth preformed flap attached to the outer layer of the stent, and wherein the first preformed flap, the second preformed flap, the third preformed flap and the fourth preformed flap are helically arranged around the tubular scaffold.

Another example stent includes a tubular scaffold, the scaffold including an inner surface, an outer surface and a lumen extending therein. The stent also includes an outer layer disposed along the outer surface of the tubular scaffold and a plurality of preformed flaps formed separated from and attached to the outer layer, wherein each of the preformed flaps is configured to pivot between a radially extended position and a collapsed position. Additionally, each of the plurality of preformed flaps is configured to releasably engage a vessel wall when the stent is implanted therein.

Alternatively or additionally to any of the embodiments above, wherein each of the plurality of preformed flaps is configured to deflect radially inward from a radially extended position.

Alternatively or additionally to any of the embodiments above, wherein each of the plurality of preformed flaps is bonded to the outer layer using a bonding material.

Alternatively or additionally to any of the embodiments above, wherein the outer layer, the plurality of preformed flaps and the bonding material are formed from the same material.

Alternatively or additionally to any of the embodiments above, wherein the plurality of preformed flaps are uniformly spaced around a circumference of the tubular scaffold.

Alternatively or additionally to any of the embodiments above, wherein plurality of preformed flaps are longitudinally aligned along the tubular scaffold.

Alternatively or additionally to any of the embodiments above, wherein the plurality of preformed flaps are helically arranged around the tubular scaffold.

Another example stent includes an expandable tubular scaffold having an outer surface and an opposite inner surface, the tubular scaffold including a first end region, a second end region and a medial region positioned between the first and second end regions. The stent also includes an outer layer disposed along the outer surface of the tubular scaffold and a plurality of preformed flaps attached to the outer layer and extending outward therefrom, wherein each of the preformed flaps is configured to pivot between a radially extended position and a collapsed position. Additionally, a first preformed flap of the plurality of flaps is positioned along the first end region and a second flap of the plurality of flaps is positioned along the second end region. Additionally, each of the plurality of preformed flaps is configured to releasably engage a vessel wall when the stent is implanted therein.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1A illustrates an example stent having anti-migration flaps in an extended configuration;

FIG. 1B illustrates an example anti-migration flap;

FIG. 1C illustrates the example stent of FIG. 1A whereby the anti-migration flaps are deflected radially inward;

FIG. 2 illustrates another example stent including flexible anti-migration flaps;

FIG. 3 illustrates another example stent including flexible anti-migration flaps;

FIG. 4 is a top view of the example stent shown in FIG. 3;

FIG. 5A illustrates an example flexible anti-migration flap;

FIG. 5B illustrates another example flexible anti-migration flap;

FIG. 5C illustrates another example flexible anti-migration flap;

FIG. 5D illustrates another example flexible anti-migration flap;

FIG. 6 is a top view of another example stent including flexible anti-migration flaps;

FIG. 7 illustrates another example stent including flexible anti-migration flaps;

FIG. 8 illustrates another example stent including flexible anti-migration flaps;

FIG. 9 illustrates another example stent including flexible anti-migration flaps;

FIG. 10A is a cross-sectional view taken along line 10A-10A of FIG. 9;

FIG. 10B is a cross-sectional view taken along line 10B-10B of FIG. 9;

FIG. 11 illustrates another example stent including flexible anti-migration flaps;

FIG. 12 a flexible anti-migration flap ring;

FIG. 13 illustrates an example stent including the anti-migration flap ring shown in FIG. 12;

FIG. 14 illustrates another example stent including flexible anti-migration flaps;

FIG. 15 illustrates another example stent including flexible anti-migration flaps;

FIG. 16 illustrates another example stent including flexible anti-migration flaps;

FIG. 17 illustrates another example stent including flexible anti-migration flaps.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

One possibility to reduce stent migration while also allowing a stent to be removed or re-positioned post-deployment may include attaching one or more flexible anchoring members to the outer surface of the stent. The flexible members may engage the vessel wall to releasably anchor the stent in place and reduce the risk of stent migration. Further, the flexible anchoring members may be easily removed and/or re-positioned from the body lumen post-deployment. Examples of medical devices, such as stents, including flexible anchoring members are disclosed herein.

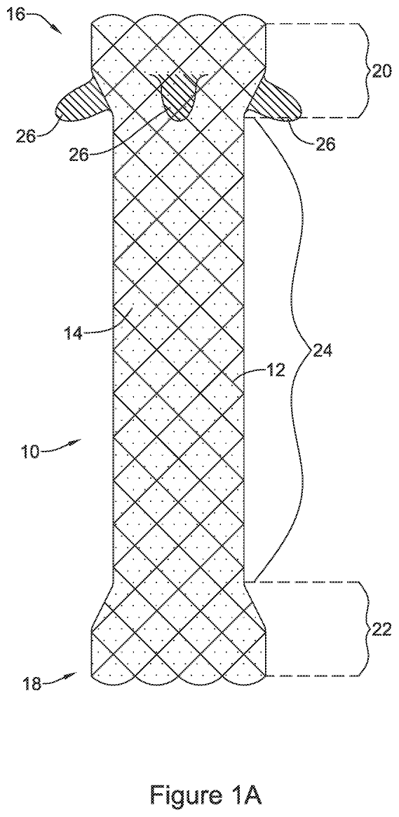

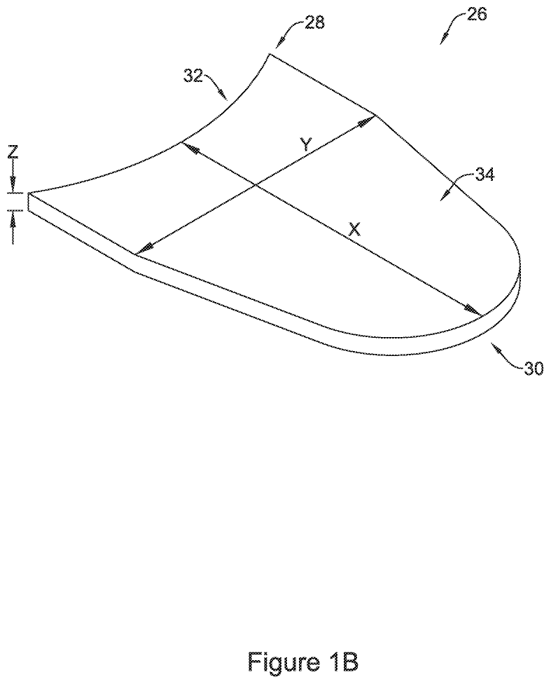

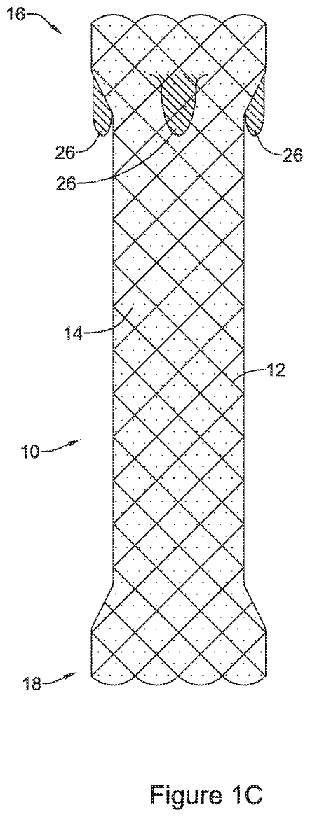

FIG. 1A shows an example stent 10. Stent 10 may have a first end 16, a second end 18 and a lumen extending therein. When positioned in a body lumen (e.g., esophagus) first or proximal end 16 may be defined as the end of stent 10 closest to a patient's mouth and second or distal end 18 may be defined as the end of stent 10 closest to a patient's stomach.

Additionally, stent 10 may include one or more stent strut members 12 forming a tubular scaffold. Stent strut members 12 may extend helically, longitudinally, circumferentially, or otherwise along stent 10. While FIG. 1A shows stent strut members 12 extending along the entire length of stent 10, in other examples, stent strut members 12 may extend only along a portion of stent 10.

FIG. 1A illustrates that, in some examples, stent 10 may include a first flared region proximate the first end 16 and/or a second flared region 22 proximate the second end 18 of stent 10. In some instances, first flared region 20 and second flared region 22 may be defined as an increase in the outer diameter, the inner diameter or both the inner and outer diameter along one or both of the first end 16 and/or second end 18 of stent 10. Further, FIG. 1A illustrates stent 10 including a medial region 24 positioned between first flared region 20 and second flared region 22.

However, it is contemplated that while FIG. 1A shows stent 10 including both a first flared region 20 and a second flared region 22, the stent 10 may only include one flared region. For example, it is contemplated that stent 10 may include only flared region 20 or flared region 22. It is further contemplated that all or a portion of first flared region 20 and/or second flared region 22 may flare outwardly (e.g., away from the central, longitudinal axis of stent 10). Alternatively, it is further contemplated that all or a portion of first flared region 20 and/or second flared region 22 may flare inwardly (e.g., toward the central, longitudinal axis of stent 10). In yet other examples, it is contemplated that stent may not include flared regions. For example, stent 10 may include a uniform diameter along its entire length.

In some instances, stent 10 may be a self-expanding stent or stent 10 may be a balloon expandable stent. Self-expanding stent examples may include stents having a tubular scaffold form of one or more filaments or struts 12 combined to form a rigid and/or semi-rigid stent structure. For example, stent filaments 12 may be wires or other filaments which are braided, wrapped, intertwined, interwoven, weaved, knitted, looped (e.g., bobbinet-style) or the like to form the tubular scaffold. For example, while the example stents disclosed herein may resemble a braided stent, this is not intended to limit the possible stent configurations. Rather, the stents depicted in the Figures may be stents including one or more, or a plurality of filaments that are knitted, braided, wrapped, intertwined, interwoven, weaved, looped (e.g., bobbinet-style) or the like to form the tubular scaffold. Alternatively, stent 10 may be a monolithic structure formed from a cylindrical tubular member, such as a single, cylindrical tubular laser-cut Nitinol tubular member, in which the remaining portions of the tubular member form the stent struts 12. Openings or interstices through the wall of the stent 10 may be defined between adjacent stent struts 12.

Stent 10 in examples disclosed herein may be constructed from a variety of materials. For example, stent 10 (e.g., self-expanding or balloon expandable) may be constructed from a metal (e.g., Nitinol, Elgiloy, etc.). In other instances, stent 10 may be constructed from a polymeric material (e.g., PET). In yet other instances, stent 10 may be constructed from a combination of metallic and polymeric materials. Additionally, stent may include a bioabsorbable and/or biodegradable material.

In some instances, example stent 10 may include one or more layers positioned on and/or adjacent to the inner and/or outer surface of the tubular scaffold of stent 10. For example, FIG. 1A shows example stent 10 including an outer layer 14 (depicted as a dotted pattern in FIG. 1A) or covering disposed along a portion of the outer surface of the tubular scaffold of the stent 10, and thus defining the outer surface of the stent 10. In some instances, outer layer 14 may be an elastomeric or non-elastomeric material. For example, outer layer 14 may be a polymeric material, such as silicone, polyurethane, or the like. The outer layer 14 (e.g., coating or covering) may be nonporous and continuous along the entire length of the tubular scaffold and extend continuously around the circumference of the tubular scaffold, and thus prevent tissue ingrowth into the central lumen of the stent 10.

As discussed herein, stents that are designed to be positioned in a body lumen (e.g., esophageal or gastrointestinal tract) may migrate due to peristalsis and/or the generally moist and inherently lubricious environment of the body lumens. Therefore, one method to reduce stent migration may include releasably attaching one or more flexible anchoring members, (e.g., flexible flaps) to the outer surface of the stent scaffold. The flexible anchoring members may provide structures that engage the vessel wall to reduce the risk of stent migration.

FIG. 1A illustrates one or more flexible anchoring members 26 positioned on the outer surface of the stent 10. The one or more anchoring members 26 may extend radially outward and away from the outer surface of the stent 10 to engage the wall of a body lumen within which stent 10 may be positioned. Further, the one or more anchoring members 26 may be designed to prevent the stent 10 from shifting or migrating with respect to a body lumen in which the stent 10 is implanted.

In some examples, the one or more flexible anchoring members 26 illustrated in FIG. 1A may be considered flexible “flaps” which are attached to the outer surface of the stent 10 to provide anti-migration capabilities. Thus, the term “flap” may be synonymous with the term “anchoring member,” as used herein. Further, the one or more flexible anchoring members 26 may be constructed as a separate component from the stent filaments or struts 12 forming the tubular scaffold and the outer layer 14, whereby the one or more anchoring members 26 may be formed separately from and subsequently attached to the outer surface of the stent 10 (e.g., attached to the outer surface of the outer layer 14) in a manufacturing step subsequent to the construction of the scaffold 12 and outer layer 14. In other words, the flexible anchoring members 26 may be “preformed” separate from the stent struts 12 and the outer layer 14. For purposes of the discussion herein, a preformed anchoring member 26 refers to anchoring members 26 (e.g., flaps) which are formed (e.g., manufactured, constructed, etc.) away from the stent 10 and separate from the outer layer 14 (e.g., coating or covering) and subsequently attached to the outer surface of the stent 10 (e.g., attached to the outer layer 14) in a separate manufacturing step subsequent to the construction of the anchoring member 26, the scaffold 12 and outer layer 14.

Further, the one or more anchoring members 26 may be formed separately from and subsequently attached to the outer surface (e.g., the outer layer 14) of the stent 10 along a pivot region of the respective anchoring member 26. The pivot region of a respective anchoring member 26 may be defined as that portion of the preformed anchoring member 26 around which the anchoring member 26 may rotate, deflect or flex. In other words, each of the preformed anchoring members 26 may be attached to the outer surface of the stent 10 along the pivot region of each anchoring member 26, whereby each respective anchoring member 26 may pivotally rotate, deflect or flex relative to the outer surface of the stent 10 at the pivot region.

As discussed herein, the anchoring members 26 may be considered “flaps.” A flap, as defined herein, may include a generally flat and broad structure that is attached along only one of its sides (e.g., edges) to the outer surface of a stent. Additionally, in some examples, flaps described herein may hang loosely along the outer surface of the stent 10 and may pivot, deflect, of flex along the flap's edge (e.g., pivoting region) which is attached to the outer surface of the stent 10.

FIG. 1B illustrates an example anchoring member 26. FIG. 1B illustrates that the anchoring member 26 may include a first end 28 and a second end 30. Additionally, as described herein, the anchoring member 26 may include a pivot region 32 extending along the width of the first end 28 of the anchoring member 26. The pivot region 32 may define the portion of the anchoring member 26 which is directly attached to the outer surface of the stent 10. The remaining portion of the anchoring member 26 may be unattached to the outer surface of the stent 10, such that the anchoring member 26 is only attached to the outer surface of the stent 10 by the pivot region 32 along one edge of the anchoring member 26. Thus, the second end 30 may be considered a free end of the anchoring member 26 that is movable radially toward or away from the outer surface of the stent 10 as the anchoring member pivots, deflects, or flexes along the pivot region 32.

Additionally, FIG. 1B illustrates that the anchoring member 26 may include a length “X” which may represent the distance the flap may extend radially outward from the outer surface of the stent 10 when fully extended. The length “X” may also represent the distance between the pivot region 32 along the base or first end 28 of the flap to the free end at the second end 30 of the flap. The length of the anchoring member 26 (the distance the anchoring members extend from the outer layer 14 when fully extended) may be about 0.5 mm to 16 mm, or about 1 mm to 14 mm, or about 1.5 mm to 12 mm, or about 2 mm to 10 mm, or about 3 mm to 8 mm, or about 4 mm to 6 mm, for example. Further, FIG. 1B illustrates that the anchoring member 26 may include a width “Y” which may represent a width of the anchoring member 26 as measured along a line taken substantially perpendicular to the length X. The width Y of the anchoring member 26 may be about 1 mm to 8 mm, or about 1.5 mm to 7 mm, or about 2 mm to 6 mm, or about 3 mm to 5 mm, for example. Further, FIG. 1B illustrates that the anchoring member 26 may include a thickness “Z”. The thickness Z may be about 5 to 120 microns, about 10 to 110 microns, 20 to 100 microns, 30 to 90 microns or about 40 to 80 microns. Thus, the thickness Z may be substantially less than the length and width of the anchoring member 26. For example, the length W and/or the width W may be at least 50 times or more, 100 times or more, 150 times or more, 200 times or more, 300 times or more of the thickness Z. Additionally, FIG. 1B illustrates that the top surface and the bottom surface of the anchoring member 26 may include a surface area 34. The surface area 34 may be about 0.5 mm² to 128 mm², or about 5 mm² to 110 mm², or about 15 mm² to 90 mm², or about 25 mm² to 70 mm², or about 35 mm² to 50 mm², for example.

In some examples, the one or more anchoring members 26 may be constructed from the same material as the outer layer 14. For example, both the one more anchoring members 26 and the outer layer 14 may include a silicone or other biocompatible polymer material. Example suitable materials which may be used to construct the one or more anchoring members 26 are listed herein. Additionally, the one or more anchoring members 26 may be bonded (e.g., secured, attached, coupled, affixed, etc.) to the outer layer 14 and/or the stent struts 12 using a bonding material constructed from the same material used to construct both the one or more anchoring members 26 and/or the outer layer 14. However, in other examples, the one more anchoring members 26, the stent struts 12 and/or the outer layer 14 may be constructed from different materials. Further, in some examples, the material utilized to bond the one or more anchoring members 26 to the outer surface of the stent 10 may be different from the material utilized to construct the one or more anchoring members 26, the stent struts 12 and/or the outer layer 14.

FIG. 1A illustrates that the one or more anchoring members 26 may be positioned along the proximal end region 16 of stent 10. For example, FIG. 1A shows the anchoring members 26 positioned along the flared region 20 of the stent 10. It is contemplated that anchoring members 26 may be positioned along any portion of the flared region 20.

Additionally, FIG. 1A illustrates the one or more anchoring members 26 may be spaced circumferentially around the outer surface of the stent 10. In some instances, the one or more anchoring members 26 may be uniformly spaced around the outer surface of the stent 10. However, in other instances, the one or more anchoring members 26 may be non-uniformly spaced around the outer surface of the stent 10.

Additionally, it is contemplated that the one or more anchoring members 26 of stent may extend radially away from stent 10 at a variety of angles, orientations, etc. For example, FIG. 1A shows anchoring members 26 located on the flared region 20 pointing toward the distal end 18 of stent 10. However, in some instances, it is contemplated that the one or more anchoring members 26 may also point toward the proximal end 16 of the stent 10. In yet other instances, it is contemplated that stent 10 may include one or more of the anchoring members 26 pointing toward the proximal end 16 of stent 10 while one or more of the anchoring members 26 point toward the distal end 18 of stent 10. Additionally, while FIG. 1A illustrates stent 10 including three anchoring members 26, it is contemplated that stent 10 may include 1, 2, 3, 4, 5, 6, 7, 8 or more anchoring members 26.

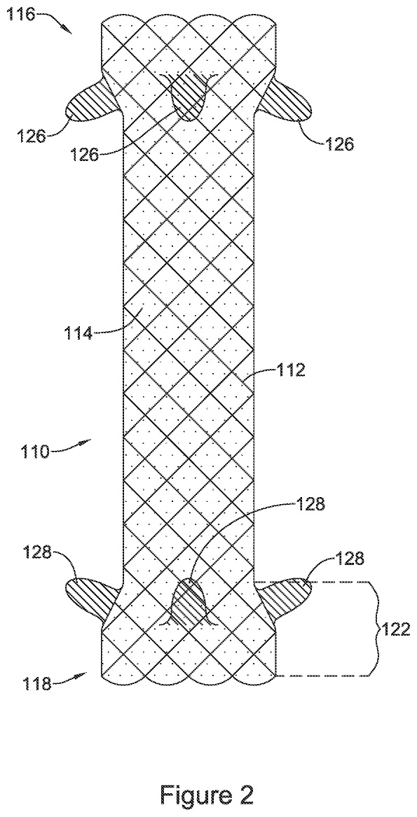

FIG. 1C illustrates the example stent 10 whereby the one or more anchoring members 26 have deflected (e.g., collapsed, flattened, flexed, pivoted, etc.) radially inward such that they are lying substantially flat against the outer surface of the stent 10. It can be appreciated that, in the configuration shown in FIG. 2, the anchoring members 26 may have disengaged from the vessel wall, thereby allowing the stent 10 to be removed from and/or repositioned within the vessel. As shown in FIG. 2, the anchoring members 26 may remain attached to the outer surface of the stent 10 as they deflect radially inward (toward the outer surface of the stent 10). The anchoring members 26 may pivot (e.g., like a hinge) along a bonded region where the anchoring member 26 attaches to the outer surface of the stent 10.

Additionally, the ability of the anchoring members 26 to deflect radially inward may be beneficial when loading the stent 10 into a stent delivery system. It can be appreciated that a stent delivery system may include a delivery catheter having a lumen designed to accept the stent 10 therein. For delivery purposes, the delivery catheter is often designed to minimize its outer diameter, and therefore, it may be beneficial to design stent 10 to be able to radially collapse to a diameter necessary to fit within the lumen of the delivery catheter. The ability of the anchoring members 26 to fold flat against the outer surface of the stent 10 is therefore beneficial in permitting the stent 10 to radially collapse into a delivery configuration.

FIG. 2 illustrates another example stent 110. Stent 110 may be similar in form and function to other stents disclosed herein. The stent 110 may include a proximal end 116, a distal end 118, stent struts 112, an outer layer 114 and anchoring members 126 positioned along the distal end region 118.

FIG. 2 further illustrates that the one or more anchoring members 128 may be positioned along the distal end region 118 of stent 110. For example, FIG. 2 shows the anchoring members 128 positioned along the flared region 122 of the stent 110. It is contemplated that anchoring members 128 may be positioned along any portion of the flared region 122.

Additionally, FIG. 2 illustrates the one or more anchoring members 128 may be spaced circumferentially around the outer surface of the stent 110. In some instances, the one or more anchoring members 128 may be uniformly spaced around the outer surface of the stent 110. However, in other instances, the one or more anchoring members 128 may be non-uniformly spaced around the outer surface of the stent 110.

Additionally, it is contemplated that the one or more anchoring members 128 of stent 110 may extend radially away from stent 110 at a variety of angles, orientations, etc. For example, FIG. 2 shows anchoring members 128 located on the flared region 122 pointing toward the proximal end 116 of stent 110. However, in some instances, it is contemplated that the one or more anchoring members 128 may also point toward the distal end 118 of the stent 110. In yet other instances, it is contemplated that stent 10 may include one or more of the anchoring members 128 pointing toward the proximal end 116 of stent 110 while one or more of the anchoring members 128 point toward the distal end 118 of stent 110. Additionally, while FIG. 2 illustrates stent 110 including three anchoring members 126, it is contemplated that stent 110 may include 1, 2, 3, 4, 5, 6, 7, 8 or more anchoring members 126.

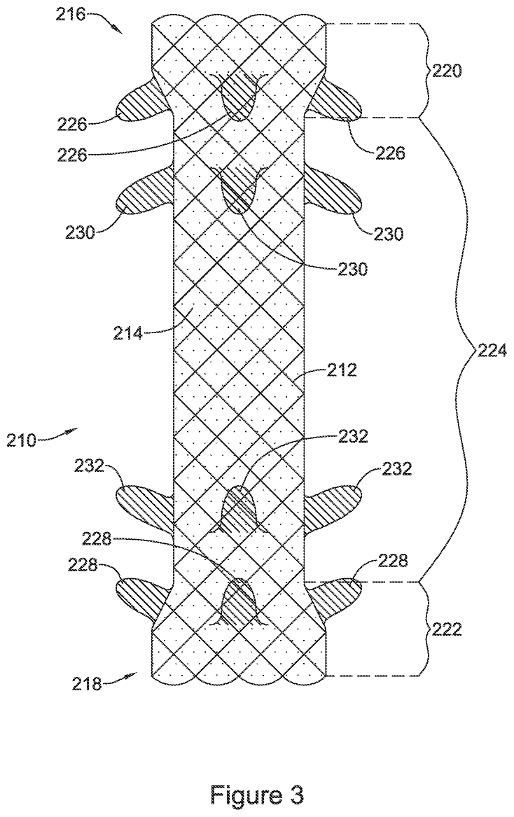

FIG. 3 illustrates another example stent 210. Stent 210 may be similar in form and function to other stents disclosed herein. The stent 210 may include a proximal end 216, a distal end 218, stent struts 212, an outer layer 214, anchoring members 226 positioned along the flared region 220 of distal end region 218 and anchoring members 228 positioned along the flared region 222 of distal end region 218.

FIG. 3 further illustrates that the stent 210 may further include one or more anchoring members 230 positioned along the medial region 224 of stent 210. The anchoring members 230 may be positioned distal to the anchoring members 226. The anchoring members 230 may extend radially away from stent 210 at a variety of angles, orientations, etc. For example, FIG. 3 shows anchoring members 230 located on the medial region 224 and pointing toward the distal end 218 of stent 210. However, it is contemplated that the anchoring members 230 may point toward the proximal end 216 of the stent 210.

Additionally, FIG. 3 illustrates the one or more anchoring members 230 may be spaced circumferentially around the outer surface of the stent 210. In some instances, the one or more anchoring members 230 may be uniformly spaced around the outer surface of the stent 210. However, in other instances, the one or more anchoring members 230 may be non-uniformly spaced around the outer surface of the stent 210.

Additionally, FIG. 3 further illustrates that the stent 210 may further include one or more anchoring members 232 positioned along the medial region 224 of stent 210. The anchoring members 232 may be positioned proximal to the anchoring members 228. The anchoring members 232 may extend radially away from stent 210 at a variety of angles, orientations, etc. For example, FIG. 3 shows anchoring members 232 located on the medial region 224 and pointing toward the proximal end 216 of stent 210. However, it is contemplated that the anchoring members 232 may point toward the distal end 218 of the stent 210.

Additionally, FIG. 3 illustrates the one or more anchoring members 232 may be spaced circumferentially around the outer surface of the stent 210. In some instances, the one or more anchoring members 232 may be uniformly spaced around the outer surface of the stent 210. However, in other instances, the one or more anchoring members 232 may be non-uniformly spaced around the outer surface of the stent 210.

It is contemplated that various combinations of the anchoring members 226, 228, 230, 232 may point toward the proximal end 216 and the distal end 218 of stent 210. For example, one or more of the anchoring members 226, 228, 230, 232 may point toward the proximal end 216 and one or more of the anchoring members 226, 228, 230, 232 may point toward the distal end 218 of stent 210. Additionally, while FIG. 3 illustrates stent 210 including twelve anchoring members 226, it is contemplated that stent 210 may include 1, 2, 3, 4, 5, 6, 7, 8, 9, 10, 11, 12, 13, 14, 15 or more anchoring members 226.

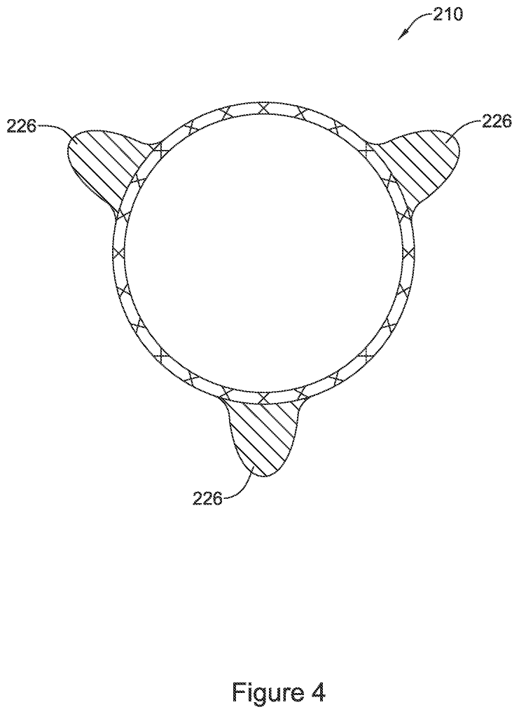

FIG. 4 illustrates a top view of the stent 210. Specifically, FIG. 4 illustrates that the anchoring members 226 may extend radially away from the outer surface of the stent 210. Further, FIG. 4 illustrates that the anchoring members 226 may be uniformly spaced around the outer surface of the stent 210. While FIG. 4 illustrates the anchoring members 226, the anchoring members 228, 230, 232 may also extend radially away from the outer surface of the stent 210 and be uniformly spaced around the outer surface of the stent 210 (similar to the anchoring members 226).

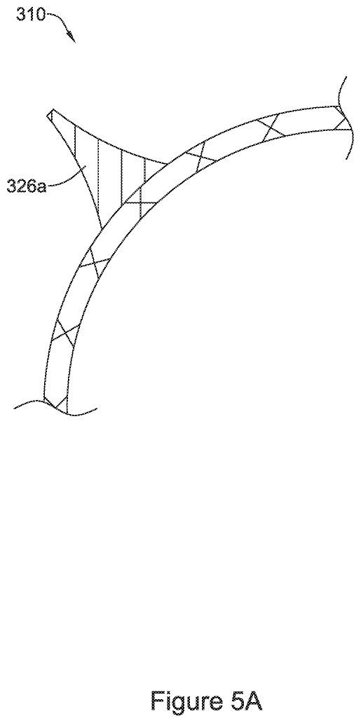

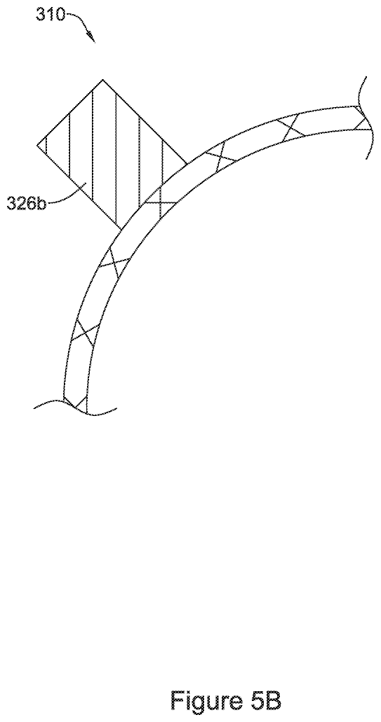

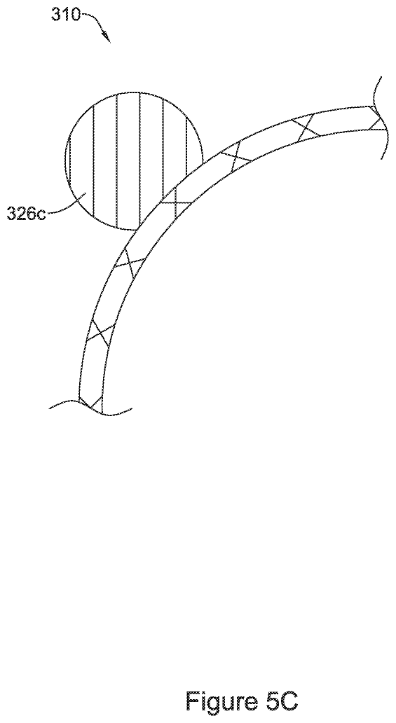

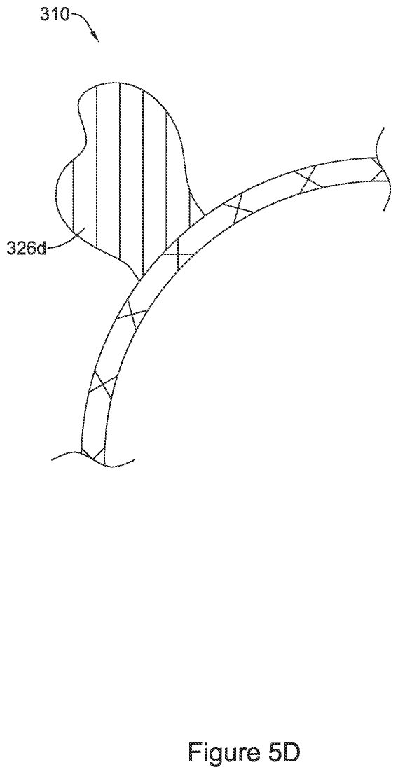

Additionally, FIG. 4 illustrates that any of the anchoring members described herein may include a variety of shapes. For example, FIG. 4 illustrates that the anchoring members 226 may include a generally ear-shaped flap extending away from the outer surface of the stent 210. Additional example shapes for the flexible anchoring members are illustrated in FIGS. 5A-5D. For example, FIG. 5A illustrates an example stent 310 (the example stent 310 may be similar in form and function to other stents disclosed herein) including an example anchoring member 326a having a generally triangular shape extending radially away from the outer surface of stent 310. FIG. 5B illustrates an example anchoring member 326b may include a generally rectangular shape extending radially away from the outer surface of the example stent 310. FIG. 5C illustrates an example anchoring member 326c may include a generally circular shape extending radially away from the outer surface of the example stent 310. FIG. 5D illustrates an example anchoring member 326d may include a generally irregular, non-symmetrical shape extending radially away from the outer surface of the example stent 310.

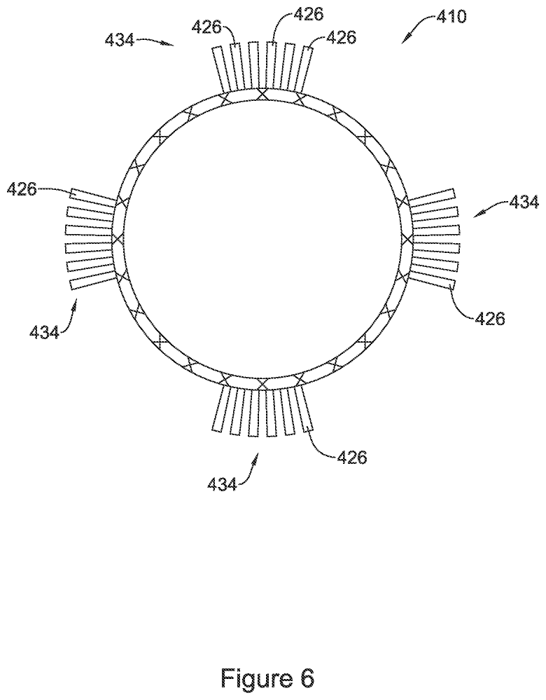

Additionally, FIG. 6 illustrates a top view of another example stent 410 which may be similar in form and function to other stents disclosed herein. FIG. 6 illustrates that the example stent 410 may include a one or more “comb-like” or “fringe-like” anchoring members 426 arranged around the outer surface of the stent 410. FIG. 6 illustrates that the anchoring members 426 may resemble a plurality of narrow, tine-like projections or strips (e.g., resembling the teeth of a comb) extending radially away from the outer surface of the stent 410. Additionally, FIG. 6 illustrates that the anchoring members 426 may be discontinuous and spaced within individual groupings 434 around the circumference of the outer surface of the stent 410. However, it is contemplated that, in some examples, anchoring members 426 may extend continuously around the circumference of the outer surface of the stent 410. In other words, in some examples, the stent 410 may not include multiple anchoring member groups 434, but rather, the comb-like or fringe-like anchoring members 426 would extend continuously around the circumferential of the outer surface of the stent 410.

In some instances, it can be appreciated the anchoring members 426 may be formed as an individual component (e.g., comb-like structure or fringe-like structure) which is attached to the outer surface of the stent 410 in a manufacturing step distinct from the construction of the scaffold 412 and applying the outer layer 414 to the scaffold 412.

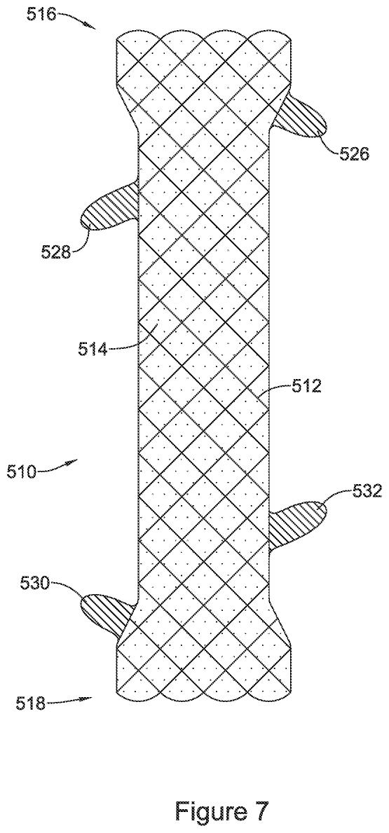

FIG. 7 illustrates another example stent 510. Stent 510 may be similar in form and function to other stents disclosed herein. The stent 510 may include a proximal end region 516, a distal end region 518, stent struts 512 and an outer layer 514.

Additionally, FIG. 7 further illustrates that the stent 510 may include anchoring members 526, 528 positioned along a proximal end region 516 of the stent 510. However, FIG. 7 illustrates that the anchoring member 526 may be longitudinally offset from the anchoring member 528. In other words, the anchoring member 526 may be positioned closer to the proximal end of the stent 510 compared to the anchoring member 528. In some examples, the anchoring member 526 may be positioned along the flared region of the stent 510 while the anchoring member 528 may be positioned along the medial region of the stent 510.

Additionally, FIG. 7 further illustrates that the stent 510 may include anchoring members 530, 532 positioned along a distal end region 518 of the stent 510. However, FIG. 7 illustrates that the anchoring member 530 may be longitudinal offset from the anchoring member 532. For example, anchoring member 530 may be positioned closer to the distal end of the stent 510 compared to the anchoring member 532. In some examples, the anchoring member 530 may be positioned along the flared region of the stent 510 while the anchoring member 532 may be positioned along the medial region of the stent 510.

The anchoring members 526, 528 may extend radially away from stent 510 at a variety of angles, orientations, etc. For example, FIG. 7 shows anchoring members 526, 528 pointing toward the distal end 518 of stent 510. However, it is contemplated that the anchoring members 528, 526 may point toward the proximal end 516 of the stent 510.

The anchoring members 530, 532 may extend radially away from stent 510 at a variety of angles, orientations, etc. For example, FIG. 7 shows anchoring members 530, 532 pointing toward the proximal end 516 of stent 510. However, it is contemplated that the anchoring members 530, 532 may point toward the distal end 518 of the stent 510.

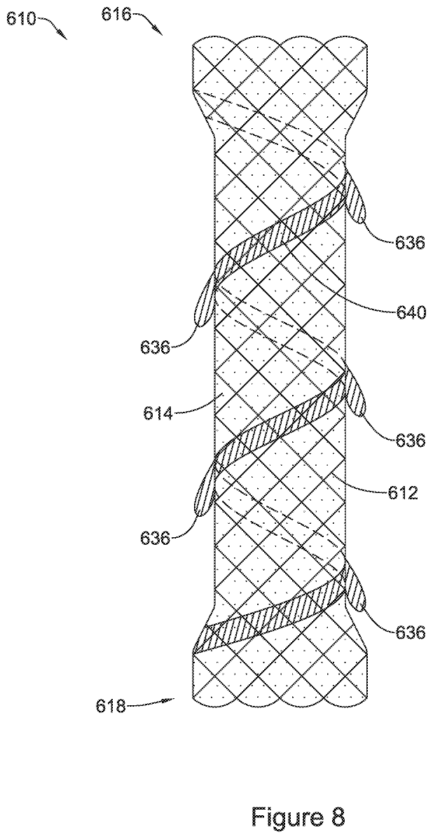

FIG. 8 illustrates another example stent 610. Stent 610 may be similar in form and function to other stents disclosed herein. The stent 610 may include a proximal end region 616, a distal end region 616, stent struts 616 and an outer layer 614.

FIG. 8 further illustrates that the stent 610 may include a one or more anchoring members 636 coupled to a spiral base member 640 (e.g. a spiral ridge) The base member 640 may generally be attached to the outer surface of the stent 610 (e.g., attached to the outer layer 614). Additionally, as illustrated in FIG. 8, the base member 640 may helically wrap around the outer surface of the stent 610. In some examples, the anchoring members 636 may form a monolithic structure with the base member 640. In other words, the anchoring members 636 may be constructed from the same material as the base member 640. Further, in some examples, the anchoring members 636 may be cut out of the base member 640 (e.g., a portion of the base member 640 may be cut into the shape of the anchoring member 636 and pulled radially away from the base member 640 to form a flap as shown in FIG. 8). However, in other examples, the anchoring members 636 may be formed independently of the base member 640 and attached in a subsequent manufacturing step.

It can be appreciated from FIG. 8 that the anchoring members 636 may be positioned (e.g., attached) along any portion of the base member 640. For example, FIG. 8 illustrates that the anchoring members 636 may be positioned uniformly along the base member 640. In the configuration shown in FIG. 8, for example, the anchoring members 636 may be equally spaced circumferentially, but longitudinally offset from one another. However, in other examples, the anchoring members 636 may be non-uniformly positioned (e.g., attached) along the base member 640. It can be appreciated that when positioned non-uniformly along the base member 640, the anchoring members 636 may be both circumferentially and longitudinally offset from one another.

Additionally, it is contemplated that the base member 640 may be constructed from the same material as the outer layer 614. The base member 640 may be bonded to the outer layer using a variety of materials, including the same material used to construct the outer layer 614.

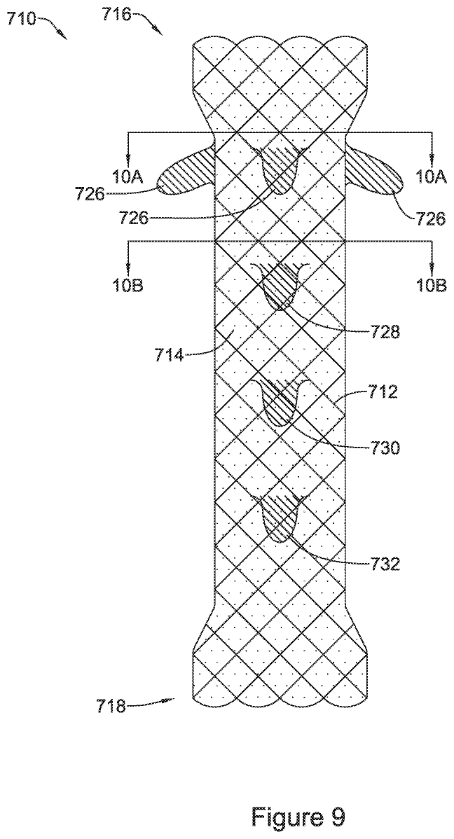

FIG. 9 illustrates another example stent 710. Stent 710 may be similar in form and function to other stents disclosed herein. The stent 710 may include a proximal end region 716, a distal end region 718, stent struts 712, an outer layer 714 and anchoring members 726 positioned along the distal end region 718.

FIG. 9 illustrates that the stent 710 may further include anchoring members 728, anchoring members 730 and anchoring members 732 attached to the outer surface of the stent 710. It can be appreciated from FIG. 9 that the anchoring members 728, anchoring members 730 and anchoring members 732 may be spaced apart from each other along the longitudinal axis of the stent 710. FIG. 9 further illustrates that the anchoring members 728, anchoring members 730 and anchoring members 732 may be radially aligned with one another along the circumference of the stent 710.

The anchoring members 726, 728, 730, 732 may extend radially away from stent 710 at a variety of angles, orientations, etc. For example, FIG. 9 shows anchoring members 726, 728, 730, 732 pointing toward the distal end 718 of stent 710. However, it is contemplated that the anchoring members 726, 728, 730, 732 may point toward the proximal end 716 of the stent 710.

It is contemplated that various combinations of the anchoring members 726, 728, 730, 732 may point toward the proximal end 716 and the distal end 718 of stent 710. For example, one or more of the anchoring members 726, 728, 730, 732 may point toward the proximal end 716 and one or more of the anchoring members 726, 728, 730, 732 may point toward the distal end 718 of stent 710.

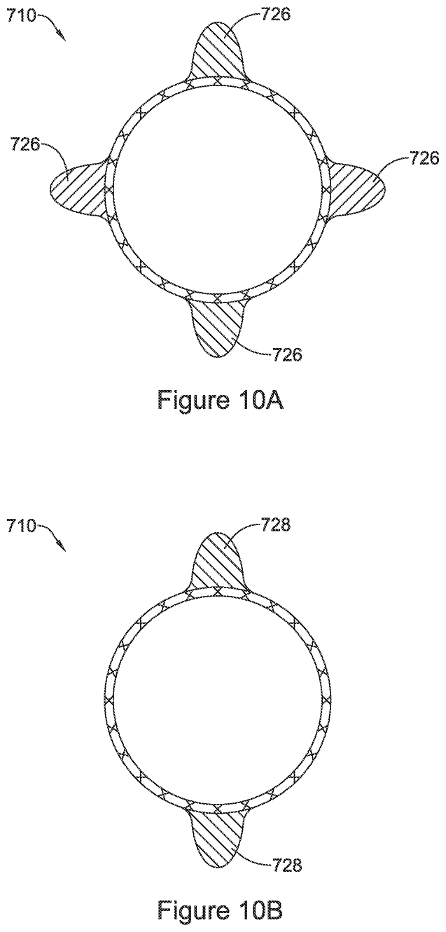

Further, it can be appreciated that additional anchoring members 726, 728, 730, 732 may be positioned on stent 710, but are hidden from view in FIG. 9. The additional anchoring members 726, 728, 730, 732 are described with respect to FIGS. 10A-10B herein.

For example, FIG. 10A is a cross-sectional view taken along line 10A-10A of FIG. 9. FIG. 10A illustrates four anchoring members 726 uniformly spaced around the outer circumference of the stent 710. The anchoring members 726 may take on a variety of shapes, including the example shapes of other anchoring members disclosed herein.

FIG. 10B is a cross-sectional view taken along line 10B-10B of FIG. 9. FIG. 10B illustrates two anchoring members 728 circumferentially spaced 180 degrees apart from one another. It can be appreciated that one of the anchoring members 728 is visible in FIG. 9, while the other anchoring member 728 is hidden from view in FIG. 9. Additionally, it is contemplated that the anchoring members 730, 732 of stent 710 may be arranged similarly around the circumference of stent 710 as the anchoring members 728 shown in FIG. 10B. In other words, in FIG. 10B, the anchoring members 730, 732 are hidden by the anchoring members 728. However, it is contemplated that stent 710 includes two anchoring members 730 circumferentially spaced 180 degrees apart and two anchoring members 732 circumferentially spaced 180 degrees apart. Therefore, stent 710 includes four anchoring members 726 positioned along the proximal end 716, and two “vertical strips” of anchoring members 728, 730, 732 spaced 180 degrees apart and longitudinally aligned along the outer surface of the stent 710.

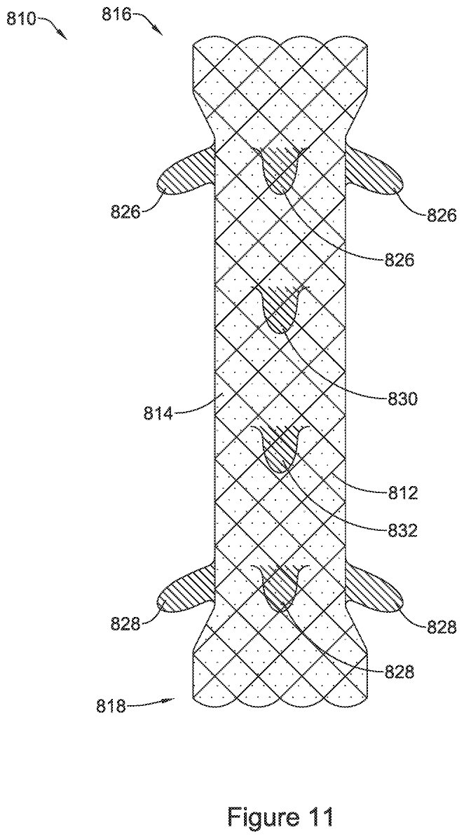

FIG. 11 illustrates another example stent 810. Stent 810 may be similar in form and function to the stent 710 described herein. For example, the stent 810 may include a proximal end region 816, a distal end region 818, stent struts 812, an outer layer 814, four anchoring members 826 positioned along the proximal end region 816, two anchoring members 828 positioned along the medial region of the stent and two anchoring members 830 positioned along the medial region of the stent. However, FIG. 11 illustrates that the stent 810 may further include four anchoring members 828 position along the distal end region 818 of the stent 810. Like the anchoring members 726 of stent 710 described above with respect to FIG. 10A, the four anchoring members 828 may be uniformly spaced around the outer surface of the stent 810.

The anchoring members 826, 828, 830, 832 may extend radially away from stent 810 at a variety of angles, orientations, etc. For example, FIG. 11 shows anchoring members 826, 828, 830, 832 pointing toward the distal end 818 of stent 810. However, it is contemplated that the anchoring members 826, 828, 830, 832 may point toward the proximal end 816 of the stent 810.

It is contemplated that various combinations of the anchoring members 826, 828, 830, 832 may point toward the proximal end 816 and the distal end 818 of stent 810. For example, one or more of the anchoring members 826, 828, 830, 832 may point toward the proximal end 816 and one or more of the anchoring members 826, 828, 830, 832 may point toward the distal end 818 of stent 810.

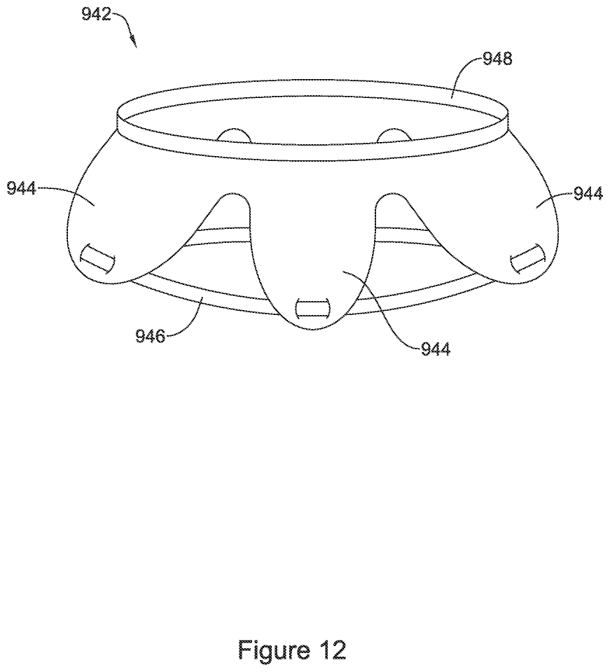

FIG. 12 illustrates an example anchoring ring 942. The anchoring ring 942 may include a plurality of anchoring members 944, whereby a base end region of each anchoring member 944 is attached to or located at the circumferential band 948. In some examples, the circumferential band 948 may be radially compressible and/or radially expandable, and/or able to be cinched to a smaller diameter. For example, in some examples, the circumferential band 948 may be constructed from an elastic material. Accordingly, the circumferential band 948 may be able to be radially stretched to dispose the anchoring ring 942 around a region of the tubular scaffold of the stent. The circumferential band 948 may be attached to the outer surface of the stent. For example, the circumferential band 948 may be bonded to the outer surface of the stent. However, in other examples, the circumferential band 948 may be stitched to the outer surface of the stent.

The anchoring members 944 may be similar in form and function to other anchoring members described herein. For example, the anchoring ring 942 may include four anchoring members 944 uniformly spaced around the outer surface of the circumferential band 948. In other examples, the four anchoring members 944 may be non-uniformly spaced around the outer surface of the circumferential band 948. The anchoring members 944 may take on a variety of shapes, including the example shapes of other anchoring members disclosed herein.

The anchoring ring 942 may further include a circumferential band 946 coupled to a free end region (e.g., tip) of each of the anchoring members 944. In some examples the circumferential band 946 may be formed integral with the anchoring members 944. In other instances, the base of each anchoring member 944 may be secured to the circumferential band 946 such that the anchoring members 944 are uniformly or non-uniformly arranged around the circumference of the circumferential band 946. In some instances, the circumferential band 946 may pass through apertures positioned in the free end region of each of the anchoring members 944.

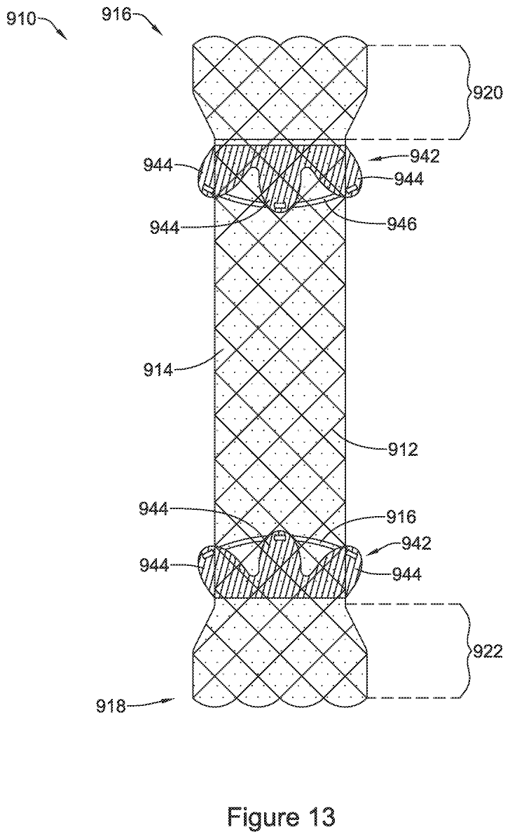

FIG. 13 illustrates another example stent 910. Stent 910 may be similar in form and function to other stents described herein. For example, the stent 910 may include a proximal end region 916, a distal end region 918, stent struts 912 and an outer layer 914.

Additionally, FIG. 13 illustrates that the stent 910 may include a first anchoring ring 942a (described above) attached distal to the proximal flared region 920 and a second anchoring ring 942b attached proximal to the distal flared region 922. It can be appreciated that each of the anchoring rings 942a, 942b may extend circumferentially around the outer surface of the stent 910. It can be further appreciated that the circumferential band 948 of each of the anchoring rings 942a, 942b may be secured to the outer surface of the stent 910. In some examples the circumferential band 948 of each of the anchoring rings 942a, 942b may be bonded, stitched or attached using any suitable attachment method. In other examples, the inner diameter of the circumferential band 948 of each of the anchoring rings 942a, 942b may be less than the outer diameter of the stent 910, and therefore, the circumferential band 948 of each of the anchoring rings 942a, 942b may be secured to the stent 910 via radial compression or a press fit along the outer surface of the stent 910. Additionally, it can be appreciated that the flared region 920 of stent 910 may act to prevent the anchoring ring 942a from translating along the stent 910 in a proximal direction, while the flared region 922 of stent 910 may act to prevent the anchoring ring 942b from translating along the stent 910 in a distal direction.

FIG. 13 further illustrates that the anchoring members 944 of the first anchoring member 942a may extend radially away from stent 910 and point toward the distal end 918 of stent 910. Similarly, FIG. 13 further illustrates that the anchoring members 944 of the second anchoring member 942b may extend radially away from stent 910 and point toward the proximal end 916 of stent 910.

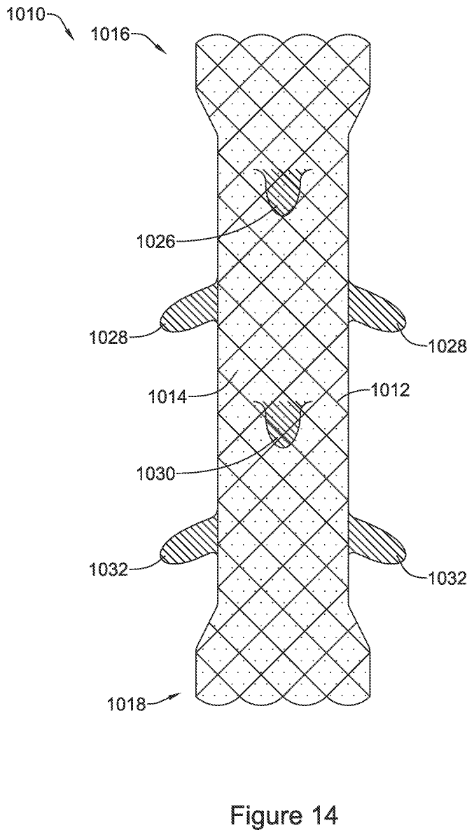

FIG. 14 illustrates another example stent 1010. Stent 1010 may be similar in form and function to other stents disclosed herein. The stent 1010 may include a proximal end region 1016, a distal end region 1018, stent struts 1012 and an outer layer 1014.

FIG. 14 further illustrates that the stent 1010 may include one or more “pairs” of anchoring members 1026, 1028, 1030, 1032 positioned along the medial region of the stent 1010. For example, FIG. 14 illustrates that the stent 1010 may include a first pair of anchoring members 1026 (one of which is hidden from view in FIG. 14, but circumferentially spaced 180 degrees from the anchoring member 1026 which is visible) positioned adjacent to the proximal end region 1016, a second pair of anchoring members 1028, each of which is circumferentially offset 90 degrees from the pair of anchoring members 1026, a third pair of anchoring members 1030 (one of which is hidden from view in FIG. 14, but circumferentially spaced 180 degrees from the anchoring member 1030 which is visible), each of which is circumferentially offset 90 degrees from the pair of anchoring members 1028 and a fourth pair of anchoring members 1032, each of which is circumferentially offset 90 degrees from the pair of anchoring members 1030. It can be appreciated that each pair of anchoring members 1026, 1028, 1030, 1032 are longitudinally offset from one another along the outer surface of the stent 1010.

The anchoring members 1026, 1028, 1030, 1032 may extend radially away from stent 101 at a variety of angles, orientations, etc. For example, FIG. 14 shows anchoring members 1026, 1028, 1030, 1032 pointing toward the distal end 1018 of stent 1010. However, it is contemplated that the anchoring members 1026, 1028, 1030, 1032 may point toward the proximal end 1016 of the stent 1010.

It is contemplated that various combinations of the anchoring members 1026, 1028, 1030, 1032 may point toward the proximal end 1016 and the distal end 1018 of stent 1010. For example, one or more of the anchoring members 1026, 1028, 1030, 1032 may point toward the proximal end 1016 and one or more of the anchoring members 1026, 1028, 1030, 1032 may point toward the distal end 1018 of stent 1010. In yet other instances, it is contemplated that stent 1010 may include one or more of the anchoring members 1026, 1028, 1030, 1032 pointing toward the proximal end 1016 of stent 1010 while one or more of the anchoring members 1026, 1028, 1030, 1032 point toward the distal end 1018 of stent 1010.

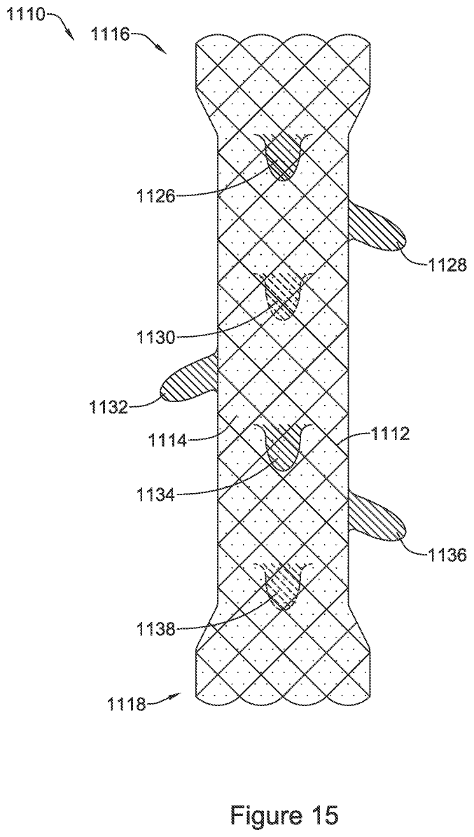

FIG. 15 illustrates another example stent 1110. Stent 1110 may be similar in form and function to other stents disclosed herein. The stent 1110 may include a proximal end region 1116, a distal end region 1118, stent struts 1112 and an outer layer 1114.

FIG. 15 further illustrates that the stent 1110 may include a plurality of anchoring members 1126, 1128, 1130, 1132, 1134, 1136, 1138 attached to the outer surface of the medial region of the stent 1110. Additionally, FIG. 15 illustrates that the anchoring members 1126, 1128, 1130, 1132, 1134, 1136 may be arranged in a helical orientation whereby each of the anchoring members 1126, 1128, 1130, 1132, 1134, 1136, 1138 are circumferentially offset from one another by 90 degrees. For example, FIG. 15 illustrates that the proximal most anchoring member 1126 may be positioned adjacent the proximal end region 1116 of stent 1110, the anchoring member 1128 may be positioned distal to the anchoring member 1126 and offset 90 degrees therefrom, the anchoring member 1128 may be positioned distal to the anchoring member 1126 and offset 90 degrees therefrom, the anchoring member 1130 may be positioned distal to the anchoring member 1128 and offset 90 degrees therefrom, the anchoring member 1132 may be positioned distal to the anchoring member 1130 and offset 90 degrees therefrom, the anchoring member 1134 may be positioned distal to the anchoring member 1132 and offset 90 degrees therefrom, the anchoring member 1136 may be positioned distal to the anchoring member 1134 and offset 90 degrees therefrom and the anchoring member 1138 may be positioned distal to the anchoring member 1136 and offset 90 degrees therefrom.

The anchoring members 1126, 1128, 1130, 1132, 1134, 1136, 1138 may extend radially away from stent 1110 at a variety of angles, orientations, etc. For example, FIG. illustrates anchoring members 1126, 1128, 1130, 1132, 1134, 1136, 1138 pointing toward the distal end 1118 of stent 1110. However, it is contemplated that the anchoring members 1126, 1128, 1130, 1132, 1134, 1136, 1138 may point toward the proximal end 1116 of the stent 1110.

It is contemplated that, in some examples, stent 1110 may include one or more of the anchoring members 1126, 1128, 1130, 1132, 1134, 1136, 1138 pointing toward the proximal end 1116 of stent 1110 while one or more of the anchoring members 1126, 1128, 1130, 1132, 1134, 1136, 1138 point toward the distal end 1118 of stent 1110. In yet other instances, it is contemplated that stent 1110 may include one or more of the anchoring members 1126, 1128, 1130, 1132, 1134, 1136, 1138 pointing toward the proximal end 1116 of stent 1100 while one or more of the anchoring members 1126, 1128, 1130, 1132, 1134, 1136, 1138 point toward the distal end 1118 of stent 1110.

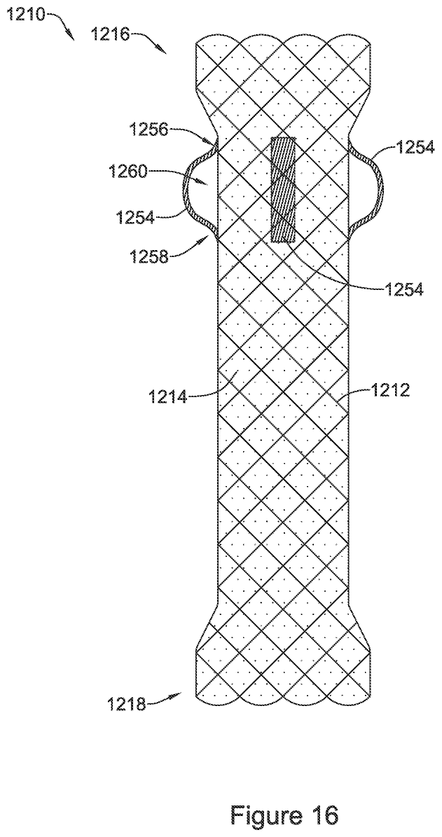

FIG. 16 illustrates another example stent 1210. Stent 1210 may be similar in form and function to other stents disclosed herein. The stent 1210 may include a proximal end region 1216, a distal end region 1218, stent struts 1212 and an outer layer 1214.

Additionally, FIG. 16 illustrates that stent 1210 may include one or more anchoring members 1254. In some examples, the anchoring members 1254 may be uniformly arranged around the circumference of the stent 1210. For example, while three anchoring members 1254 are shown in FIG. 16, it can be appreciated that a fourth anchoring member 1254 (hidden from view) is included in stent 1210. The fourth anchoring member 1254 may be spaced equidistant from the other anchoring members 1254 shown in FIG. 16. Additionally, while FIG. 16 is described herein as including four anchoring members 1254, it is contemplated that stent 1210 may include 1, 2, 3, 4, 5, 6, 7, 8 or more anchoring members 1254.

Each of the anchoring members 1254 may include a first end 1256 and a second end 1258. As illustrated in FIG. 16, both the first end 1256 and the second end 1258 may be attached to the outer surface of the stent 1210. Additionally, FIG. 16 illustrates that a medial portion of each anchoring member 1254 may extend radially outward from the outer surface of the stent 1210 to create an aperture 2160 (e.g., opening, cavity, etc.) between an inner surface of the anchoring member 1254 and the outer surface of the stent 1254. It can be appreciated that aperture 1254 may provide a space for tissue ingrowth, thereby releasably engaging the stent 1210 to the vessel wall. It can be further appreciated that while FIG. 16 illustrates the anchoring members aligned along the longitudinal axis of the stent 1210 (e.g., vertically aligned), in some examples the anchoring members 1254 may be arranged horizontally, diagonally or any combination of vertical, horizontal or diagonal alignment along the stent 1210.

In some examples, the one or more anchoring members 1254 may be constructed from the same material as the outer layer 1214. For example, the one or more anchoring members 1254 and the outer layer 1214 may include a silicon material. Additionally, the one or more anchoring members 1254 may be bonded (e.g., secured, attached, coupled, etc.) to the outer layer 1214 and/or the stent struts 1212 using a bonding material constructed from the same material used to construct both the one or more anchoring members 1254 and/or the outer layer 1214. However, in other examples, the one more anchoring members 1254, the stent struts 1212 and/or the outer layer 1214 may be constructed from different materials. Further, in some examples, the material utilized to bond the one or more anchoring members 1254 to the outer surface of the stent 1210 may be different from the material utilized to construct the one or more anchoring members 1254, the stent struts 1212 and/or the outer layer 1214.

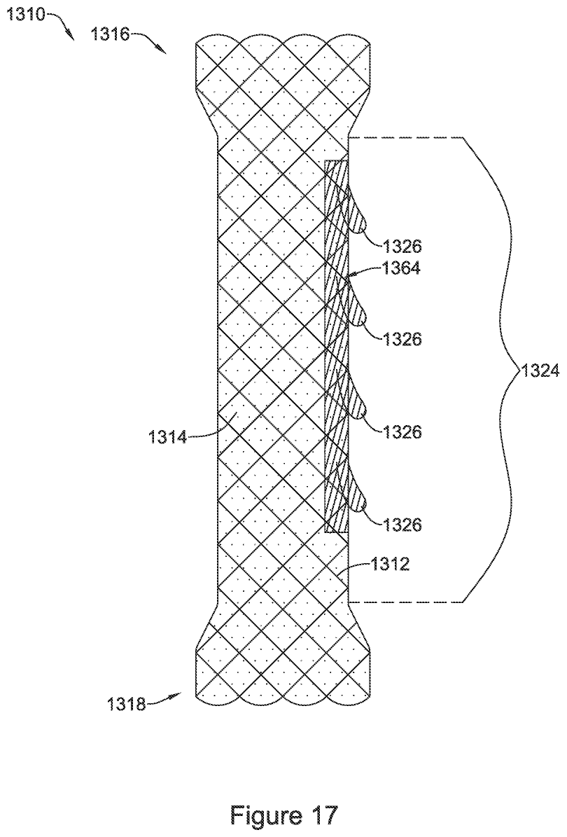

FIG. 17 illustrates another example stent 1310. Stent 1310 may be similar in form and function to other stents disclosed herein. The stent 1310 may include a proximal end region 1316, a distal end region 1316, stent struts 1316 and an outer layer 1314.

FIG. 17 further illustrates that the stent 1310 may include a one or more anchoring members 1326 coupled to a base member 1364. The base member 1364 may generally be attached to the outer surface of the stent 1310. Additionally, as illustrated in FIG. 17, the base member 1364 may extend longitudinally along the outer surface of the stent 1310. In some examples, the anchoring members 1326 may be a monolithic structure with the base member 1364. In other words, the anchoring members 1326 may be constructed from the same material as the base member 1364. Further, in some examples, the anchoring members 1326 may be cut out of the base member 1364 (e.g., a portion of the base member 1364 may be cut into the shape of the anchoring member 1326 and extended radially away from the base member 1364 to form a flap as shown in FIG. 17). However, in other examples, the anchoring members 1326 may be formed independently of the base member 1364 and attached to the base member 1364 in a subsequent manufacturing step.

It can be appreciated from FIG. 17 that the anchoring members 1326 may be positioned (e.g., attached) along any portion of the base member 1364. For example, FIG. 17 illustrates that the anchoring members 1326 may be positioned uniformly along the base member 1364. In the configuration shown in FIG. 17, for example, the anchoring members 1326 may be equally spaced and longitudinally offset from one another. However, in other examples, the anchoring members 1326 may be non-uniformly positioned (e.g., attached) along the base member 1364.

Additionally, it is contemplated that the base member 1364 and/or anchoring members 1326 may be constructed from the same material as the outer layer 1314. The base member 1364 may be bonded to the outer layer using a variety of materials, including the material used to construct the outer layer 1314.

The materials that can be used for the various components of stent 10 (and/or other stents disclosed herein) and the various medical devices disclosed herein may include those commonly associated with medical devices. For simplicity purposes, the following discussion refers to stent 10 and other components of stent 10. However, this is not intended to limit the devices and methods described herein, as the discussion may be applied to other similar medical devices disclosed herein.

Stent 10 and other components of stent 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of stent 10 and other components of stent 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of stent 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into stent 10. For example, stent 10 and other components of stent 10, or portions thereof, may be made of a material that does not substantially distort the image and create substantial artifacts (e.g., gaps in the image). Stent 10 and other components of stent 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An expandable stent, comprising:
a tubular scaffold including an inner surface, an outer surface and a lumen extending therein;
an outer layer disposed along the outer surface of the tubular scaffold; and
a first preformed flap attached to the outer layer;
wherein the preformed flap is configured to releasably engage a vessel wall when the stent is implanted therein.

2. The stent of claim 1, wherein the first preformed flap is configured to pivot between a radially extended position and a collapsed position.

3. The stent of claim 1, wherein the first preformed flap is configured to deflect radially inward from a radially extended position.

4. The stent of claim 1, wherein the first preformed flap includes a first attachment region positioned along a first edge of the flap, and wherein the first preformed flap is attached to the outer layer along the first attachment region.

5. The stent of claim 1, wherein the first preformed flap is bonded to the outer layer using a bonding material.

6. The stent of claim 5, wherein the outer layer, the first preformed flap and the bonding material are formed from the same material.

7. The stent of claim 1, further comprising a second preformed flap, and wherein the first preformed flap and the second preformed flap are uniformly spaced around the tubular scaffold.

8. The stent of claim 7, wherein the first preformed flap and the second preformed flap are longitudinally aligned with each other along the outer layer of the stent.

9. The stent of claim 7, wherein the first preformed flap and the second preformed flap are longitudinally offset from each other along the outer layer of the stent.

10. The stent of claim 1, wherein the first preformed flap is positioned along a proximal end region of the tubular scaffold, and wherein the second preformed flap is positioned along a distal end region of the tubular scaffold.

11. The stent of claim 10, wherein the first preformed flap extends in a distal direction toward a distal end of the tubular scaffold, and wherein the second preformed flap extends in a proximal direction toward a proximal end of the tubular scaffold.

12. The stent of claim 1, further comprising a second preformed flap attached to the outer layer of the stent, a third preformed flap attached to the outer layer of the stent and a fourth preformed flap attached to the outer layer of the stent, and wherein the first preformed flap, the second preformed flap, the third preformed flap and the fourth preformed flap are helically arranged around the tubular scaffold.

13. A stent, comprising:
a tubular scaffold, the scaffold including an inner surface, an outer surface and a lumen extending therein;
an outer layer disposed along the outer surface of the tubular scaffold; and
a plurality of preformed flaps formed separated from and attached to the outer layer, wherein each of the preformed flaps is configured to pivot between a radially extended position and a collapsed position;
wherein each of the plurality of preformed flaps is configured to releasably engage a vessel wall when the stent is implanted therein.

14. The stent of claim 13, wherein each of the plurality of preformed flaps is configured to deflect radially inward from a radially extended position.

15. The stent of claim 13, wherein each of the plurality of preformed flaps is bonded to the outer layer using a bonding material.

16. The stent of claim 15, wherein the outer layer, the plurality of preformed flaps and the bonding material are formed from the same material.

17. The stent of claim 13, wherein the plurality of preformed flaps are uniformly spaced around a circumference of the tubular scaffold.

18. The stent of claim 13, wherein plurality of preformed flaps are longitudinally aligned along the tubular scaffold.

19. The stent of claim 13, wherein the plurality of preformed flaps are helically arranged around the tubular scaffold.

20. A stent, comprising:
an expandable tubular scaffold having an outer surface and an opposite inner surface, the tubular scaffold including a first end region, a second end region and a medial region positioned between the first and second end regions;
an outer layer disposed along the outer surface of the tubular scaffold; and
a plurality of preformed flaps attached to the outer layer and extending outward therefrom, wherein each of the preformed flaps is configured to pivot between a radially extended position and a collapsed position;
wherein a first preformed flap of the plurality of flaps is positioned along the first end region, and wherein a second flap of the plurality of flaps is positioned along the second end region; and
wherein each of the plurality of preformed flaps is configured to releasably engage a vessel wall when the stent is implanted therein.
